(19) United States
(12) Patent Application Publication
(10) Pub. No.: US 2025/0249237 A1
(43) Pub. Date: Aug. 7, 2025

(54) ELECTRODE LEADS HAVING SUTURE ANCHORS

(71) Applicant: The Alfred E. Mann Foundation for Scientific Research, Valencia, CA (US)

(72) Inventors: William Andrew Brandt, Castaic, CA (US); Joseph L. Calderon, Santa Clarita, CA (US); Oren Gotlib, Agoura Hills, CA (US)

(73) Assignee: The Alfred E. Mann Foundation for Scientific Research, Valencia, CA (US)

(21) Appl. No.: 19/045,092

(22) Filed: Feb. 4, 2025

Related U.S. Application Data
(60) Provisional application No. 63/548,714, filed on Feb. 1, 2024.

(51) Int. Cl. A61N 1/05

(52) U.S. Cl. CPC A61N 1/0558; A61N 1/0556

(57) ABSTRACT

An electrode lead including an elongate lead body having a proximal region, a distal region and a plurality of electrical conductors, a plurality of electrically conductive contacts associated with the distal region of the lead body and electrically connected to the electrical conductors, and a suture anchor, on the distal region of the lead body, including an anchor body that defines a curved three-dimensional geometric exterior surface shape and a central axis that is coincident with a plane that passes through the anchor body, a lumen that extends through the anchor body in which a portion of the lead body is located, and at least one suture lumen that is entirely on one side of the plane and that has open ends on the exterior surface.

### CROSS-REFERENCE TO RELATED APPLICATION

This application claims the benefit of U.S. Provisional Application No. 63/548,714, filed Feb. 13, 2024, and entitled “Suture Anchor For a Stimulation Lead And Stimulation Systems Incorporating the Same,” which is incorporated herein by reference.

### BACKGROUND OF THE INVENTIONS

### 1. Field of Inventions

The present inventions relate generally to implantable medical devices such as, for example, implantable tissue stimulators.

### 2. DESCRIPTION OF THE RELATED ART

Implantable tissue stimulators, which may include an implantable pulse generator (“IPG”) and an electrode lead, are used to treat a wide variety of medical conditions. The electrode leads may include a lead body and a plurality of electrically conductive contacts, although the exact configuration of the electrode lead will depend upon the medical condition being treated.

One exemplary condition that may be treated with an implantable tissue stimulator is obstructive sleep apnea (OSA), which is a highly prevalent sleep disorder that is caused by the collapse of or increase in the resistance of the pharyngeal airway, often resulting from tongue obstruction. Nerve fascicles of the hypoglossal nerve (HGN) that innervate the intrinsic and extrinsic muscles of the tongue are stimulated in a manner that prevents retraction of the tongue, which would otherwise close the upper airway during the inspiration portion of the respiratory cycle. Here, the electrode leads frequently include a lead body and a nerve cuff with a cuff body and a plurality of electrically conductive contacts. To that end, and referring to FIG. 1, a nerve cuff 10 may be positioned around the trunk 14 of the HGN 12 and used to stimulate the muscles that anteriorly move the tongue 16 and, in particular, the fascicles of the HGN 12 that innervate the tongue protrusor muscles, such as the genioglossus 18 and/or the geniohyoid muscles 20. The nerve cuff 10 is positioned on the HGN trunk 14 at a position 22 proximal to the HGN branches 24. Although there are advantages to implanting the nerve cuff 10 at this proximal position 22, i.e., reduced surgical time and effort as well as reduced risk and trauma to the patient, it introduces the problem of inadvertently stimulating other fascicles of the HGN trunk 14 that innervate muscles in opposition to the genioglossus 18 and/or the geniohyoid muscles 20, i.e., the tongue retractor muscles, e.g., the hyoglossus 26 and styloglossus muscles 28, as well as the intrinsic muscles of the tongue 16. Accordingly, while some clinicians may desire to stimulate the HGN 12 at the HGN trunk 14, others may desire to stimulate the HGN at the GM branch 24, and some nerve cuffs are configured in such a manner that they may be positioned the HGN GM branch 24 as well as the trunk 14 in various furled states.

Other exemplary medical conditions that may be treated with tissue stimulation include, but are not limited to, chronic pain syndrome, which may be treated spinal cord stimulation, neurological disorders, which may be treated with deep brain stimulation, and slow or irregular heartbeats, which may be treated with a pacemaker. Here too, the associated tissue stimulators may include an electrode lead with a lead body and a plurality of electrically conductive contacts.

A portion of a lead body may be secured to a body structure with sutures in order to prevent unwanted movement of the electrically conductive contacts. To facilitate this process, some lead bodies include suture anchors that are used to position and maintain the sutures at the desired location on the lead bodies. The sutures extend around a portion suture anchor and around (or through) the body structure, with the ends knotted, to secure the lead body to the body structure. In the exemplary context of OSA, the lead body may include a suture anchor that is used to secure the electrode lead to the digastric tendon.

### SUMMARY

The present inventors have determined that conventional suture anchors are susceptible to improvement. For example, the present inventors have determined that the configuration of conventional suture anchors increases the likelihood of suture-based damage to the associated lead bodies. In particular, the configuration of conventional lead bodies results in sutures extend around both the lead bodies and the body structures and, accordingly, the sutures may damage the lead bodies in those instances where the sutures are tightened to the point at which they slice through the anchors. The present inventors have determined that it would be desirable for suture anchors to be configured in such a manner that the surgeon has multiple options when it comes to the manner in which the suture will be deployed.

An electrode lead in accordance with at least one of the present inventions includes an elongate lead body having a proximal region, a distal region and a plurality of electrical conductors, a plurality of electrically conductive contacts associated with the distal region of the lead body and electrically connected to the electrical conductors, and a suture anchor, on the distal region of the lead body, including an anchor body that defines a curved three-dimensional geometric exterior surface shape and a central axis that is coincident with a plane that passes through the anchor body, a lumen that extends through the anchor body in which a portion of the lead body is located, and at least one suture lumen that is entirely on one side of the plane and that has open ends on the exterior surface.

An electrode lead in accordance with at least one of the present inventions includes an elongate lead body having a proximal region, a distal region and a plurality of electrical conductors, a nerve cuff, including a nerve cuff body and a plurality of electrically conductive contacts carried by the nerve cuff body and electrically connected to the electrical conductors, associated with the distal region of the lead body, and a strain relief, associated with the lead body and the nerve cuff body, that includes a suture anchor.

An electrode lead in accordance with at least one of the present inventions includes an elongate lead body having a proximal region, a distal region and a plurality of electrical conductors, a plurality of electrically conductive contacts associated with the distal region of the lead body and electrically connected to the electrical conductors, and a suture anchor, on the distal region of the lead body, including an anchor body that defines and a central axis and an exterior surface, a lumen that extends through the anchor body in which a portion of the lead body is located, at least one groove that extends inwardly from the outer surface, at least one protuberance that extends outwardly from the outer surface, and at least one arcuate suture holder that defines a suture opening and extends outwardly from the outer surface

The present inventions also include systems with an implantable pulse generator or other implantable stimulation device in combination with such electrode leads.

### BRIEF DESCRIPTION OF THE DRAWINGS

Detailed descriptions of exemplary embodiments will be made with reference to the accompanying drawings.

FIG. 1 is a cut-away anatomical drawing of the head and neck area illustrating the muscles that control movement of the tongue, the HGN and its branches that innervate these muscles and a nerve cuff on the HGN trunk.

FIG. 2 is a plan view of a stimulation system including an electrode lead in accordance with one embodiment of a present invention.

FIG. 3 is a side view of a portion of the electrode lead illustrated in FIG. 2.

FIG. 4 is a plan view of the nerve cuff illustrated in FIG. 2 in an unfurled state.

FIG. 5 is a section view taken along line 5-5 in FIG. 2.

FIG. 6 is a section view taken along line 6-6 in FIG. 4.

FIG. 7 is a plan view of a portion of the electrode lead illustrated in FIG. 2.

FIG. 8 is a section view taken along line 8-8 in FIG. 7.

FIG. 9 is an end, cutaway view of a portion of the electrode lead illustrated in FIG. 2.

FIG. 10 is a cutaway view showing the electrode lead illustrated in FIG. 2 secured to a body structure.

FIG. 10A is a cutaway view showing the electrode lead illustrated in FIG. 2 disconnected from a body structure.

FIG. 11 is a side view of a portion of an electrode lead in accordance with one embodiment of a present invention.

FIG. 12 is a plan view of the portion of the electrode lead illustrated in FIG. 11.

FIG. 12A is an end view of the electrode lead illustrated in FIG. 11 with the suture anchor secured to a body structure and the nerve cuff around a nerve.

FIG. 13 is a side view of a portion of an electrode lead in accordance with one embodiment of a present invention.

FIG. 14 is a plan view of a portion of an electrode lead in accordance with one embodiment of a present invention.

FIG. 15 is a plan view of a portion of the electrode lead illustrated in FIG. 14.

FIG. 16 is a side view of a portion of the electrode lead illustrated in FIG. 14.

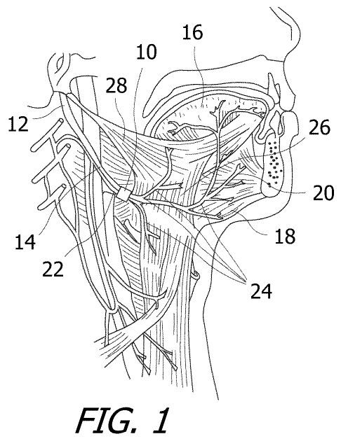

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions. For example, although described in the exemplary context of tissue stimulators and electrode leads associated with sleep apnea treatment, the present inventions are not so limited and are also applicable to implantable tissue stimulators and electrode leads that are configured to treat other medical conditions.

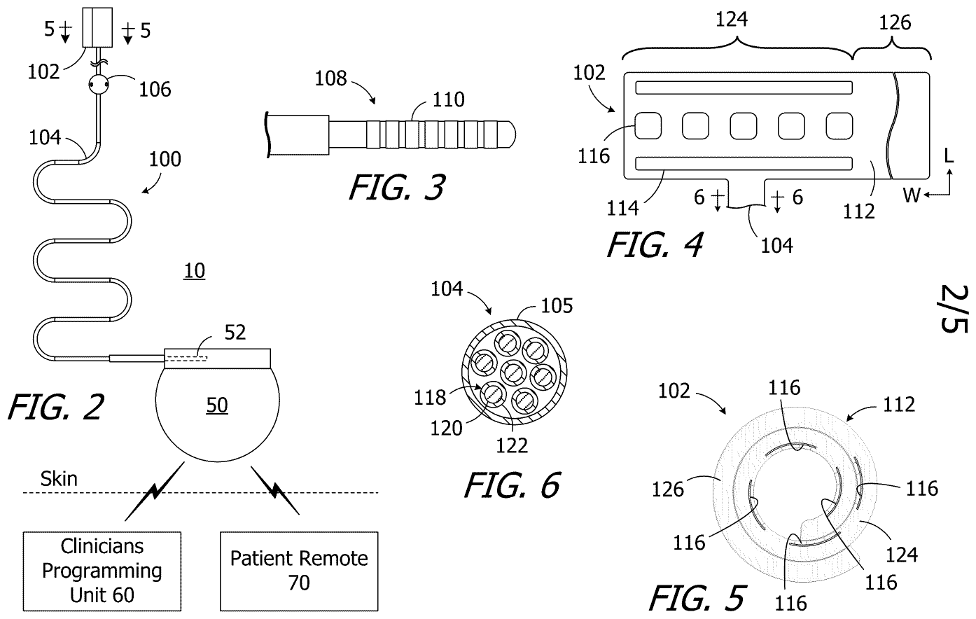

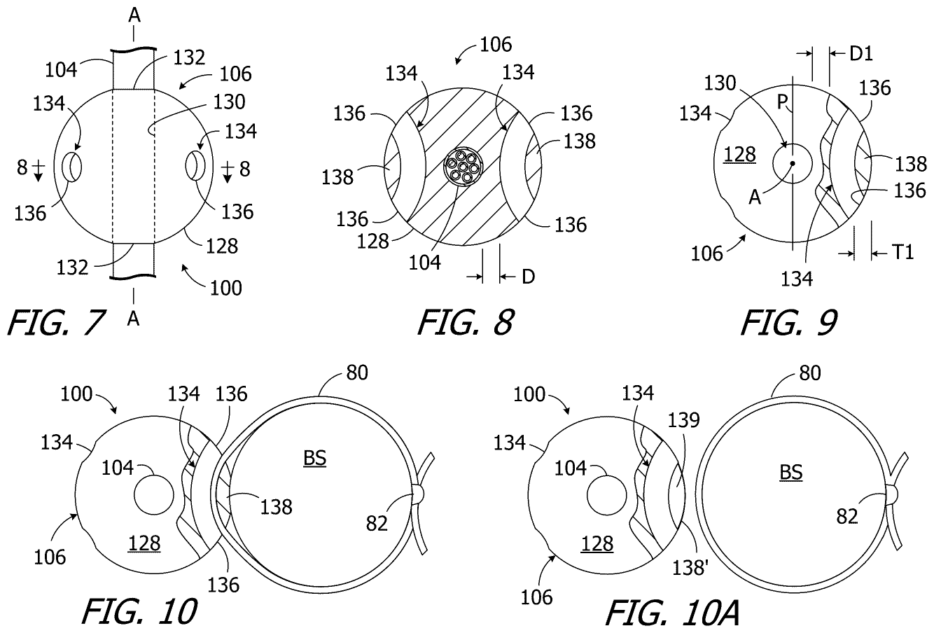

Referring to FIGS. 2-6, a stimulation system 10 in accordance with one embodiment of a present invention includes an electrode lead 100 and an implantable stimulator such as the implantable pulse generator (“IPG”) 50. A clinician's programming unit 60, a patient remote 70 and an IPG charger (not shown) may also be provided in some instances. The exemplary electrode lead 100 includes a nerve cuff 102, a lead body 104 with a proximal region and a distal region, and a suture anchor 106 on the distal region of the lead body 104. The nerve cuff 102 is discussed in greater detail below with reference to FIGS. 4-6, and the suture anchor 106 is discussed in greater detail below with reference to FIGS. 7-10A. The lead body 104 couples the nerve cuff 102 to the IPG 50 by way of a lead connector 108 with contacts 110 on the proximal end of the lead body 104 and a corresponding connector receptacle 52 on the IPG 50. The nerve cuff 102 may be pre-set (or “pre-shaped”) to the furled (or “curled”) state illustrated in FIGS. 2 and 5, and may be movable to unfurled state illustrated in FIG. 4. The exemplary lead body 104 may also include one or more S-shaped sections to provide strain relief (as shown) or may be straight. The S-shaped sections accommodate body movement at the location within the neck where the lead body 104 is implanted, thereby reducing the likelihood that the HGN will be damaged due to unavoidable pulling of the electrode lead 100 that may result from neck movements. The accommodation provided by the S-shaped sections also reduces the likelihood of fatigue damage. Additionally, although the exemplary system 10 includes a single electrode lead 100, other embodiments may include a pair of electrode leads 100 for bilateral HGN stimulation and an IPG (not shown) with two connector receptacles.

The present electrode leads may include any suitable nerve cuff configuration. By way of example, but not limitation, various exemplary nerve cuffs are illustrated and described in U.S. Pat. Pub. Nos. 2018/0318577A1, 2018/0318578A1, 2019/0060646A1, 2019/0282805A1, 2022/0062629A1, 2022/0313987A1, 2023/0010510A1, 2023/0241394A1, 2024/0009452A1, and 2024/0108883A1, which are incorporated herein by reference in their entirety, and the present leads may include any of the nerve cuffs illustrated therein.

Referring to FIGS. 4-6, the nerve cuff 102 in the illustrated embodiment includes a cuff body 112, which defines a length L and a width W that is greater than the length in the unfurled state, two relatively wide electrically conductive contacts (or “contacts”) 114 on the cuff body 112, and a plurality of relatively narrow contacts 116 on the cuff body between the contacts 114. Such contacts may also be referred to as “electrodes.” The contacts 114 and 116 may be individually electrically connected to the contacts 110 on the lead connector 108 (FIG. 2) by wires 118 (FIG. 6) that extend through the tubular member 105 of the lead body 104. Each wire 118 includes a conductor 120 and an insulator 122. The conductors 120 may be connected to the rear side of the contacts by welding or other suitable processes. The cables or other electrical conductors may be employed in place of the wires 118 in other implementations.

The cuff body 112 includes a stimulation region 124 and a compression region 126. The contacts 114 and 116 are located within the stimulation region 124, and there are no contacts located within the compression region 126. The compression region 126 wraps around at least a portion of the stimulation region 124 when the nerve cuff 102 is in the pre-shaped furled state and slightly larger, expanded and less tightly furled states, thereby resisting (but not preventing) expansion of the stimulation region and improving the electrical connection between the contacts 114 and 116 and the HGN. Suitable cuff body materials include materials that are biologically compatible, electrically insulative, elastic and capable of functioning in the manner described herein. By way of example, but not limitation, suitable cuff body materials include silicone, polyurethane and styrene-isobutylene-styrene (SIBS) elastomers. The cuff body materials should be pliable enough to allow a clinician to hold the cuff body 112 (and nerve cuff 102) in an unfurled state when the nerve cuff 102 is being placed around the HGN trunk (or HGN GM branch), yet resilient enough to cause the nerve cuff return 102 to the pre-shaped furled state illustrated in FIG. 5 when released. The materials should also be flexible enough to allow the cuff body 112 (and nerve cuff 102) to instead assume the slightly larger, expanded and less tightly furled, furled states.

Although the present inventions are not so limited, the contacts 114 are the same size and are same shape, i.e., rectangles with rounded corners, while the contacts 116 are also the same size are same shape, i.e., squares with rounded corners. In other implementations, the contacts within a particular nerve cuff may differ in shape, size, and/or orientation. Other exemplary contact shapes include, but are not limited to, rounded rectangles, circles, ovals, and squares. Suitable materials for the contacts 114 and 116 include, but are not limited to, platinum-iridium and palladium.

Turning to FIGS. 7-9, the exemplary suture anchor 106 includes an anchor body 128, a central (or “longitudinal”) axis A defined by the anchor body 128, a lumen 130 for the lead body 104 that extends through the anchor body 128 and defines open ends 132, and one or more suture lumens 134 (two in the exemplary embodiment) that define open ends 136 that are separated from one another along the outer surface of the anchor body 128. Tissue contact portions 138 are located between the open ends 136 of each suture lumen 134 and between the outer surface of the anchor body 128 and the suture lumens 134.

Referring more specifically to FIG. 9, the central axis A lies in a plane P (i.e., is coincident with the plane P) that, in the illustrated symmetrical embodiment, divides the anchor body 128 (and suture anchor 106) in half. The suture lumens 134 are located on respective opposite sides of the plane P, and each suture lumen 134 is entirely located within one of the halve of the anchor body 128 that are defined by the plane P. Each suture lumen 134 is also separated from the lumen 130 (and lead body 104) by a distance D1, which may be about 6.9 mm in the illustrated implementation. The separation reduces the likelihood that the curved needle used to thread a suture through the suture lumen will puncture the lead body 104. The tissue contact portion 138, in a plane perpendicular to the central axis A that bisects the suture lumens 134, has a thickness T1. Thickness T1 may equal to, or substantially equal to, the distance D1 and, in the illustrated embodiment, is about 6.9 mm. The thicker the tissue contact portion 138, the less likely a suture will tear through the tissue contact portion 138. As used herein in the context of distance and thickness, the word “about” means+10%.

The suture anchor 106 may be located any suitable distance from the nerve cuff 102. For example, the suture anchor 106 may be located 40 mm to 70 mm from the nerve cuff 102, and is 50 mm in the illustrated implementation, which allows the nerve cuff 102 and anchor 106 to be positioned at the desired location on a body structure such as, for example, a tendon (e.g., the digastric tendon) or muscle in the manner described below with reference to FIG. 10.

The anchor body 128 may have any suitable curved three-dimensional geometric exterior shape, and may also have a smooth outer surface, in order to limit tissue trauma. In the illustrated implementation, the anchor body 128 is spherical in shape. Put another way, the outer surface of the anchor body defines a sphere except at the ends 132 of the lumen 130. Other suitable shapes include, but are not limited to, oblate spheroids, prolate spheroids, and capsules (i.e., cylinders with hemispherical ends). The exemplary anchor body 128 also defines a central axis A and may be symmetrical (as shown) or asymmetrical about the central axis A. Additionally, the lumen 130 may be coaxial with the central axis A (as shown), or may define an axis that is offset from the central axis A. The suture lumens 134 (and open ends 136) in the exemplary embodiment are on opposite sides of the lead body 104 and, more specifically, the mid-points of suture lumens 134 (between the open ends 136) are offset by 180 degrees around the axis A. The suture lumens 134 may be identical in shape and size (as shown) or may be different in size and/or shape. The suture lumens 134 may also be curved (e.g., the illustrated minor arc) or straight. In the illustrated implementation, the suture lumens 134 are curved in a plane perpendicular to the central axis A (note FIG. 8).

One exemplary method of anchoring the exemplary electrode lead 100 is illustrated in FIG. 10. The suture anchor 106 and corresponding portion of the lead body 104 are positioned adjacent to a body structure BS (e.g., the digastric tendon). The suture anchor 106 is oriented is such a manner that one of the suture lumens 134 faces the body structure BS and the associated contact portion 138 abuts the body structure. A suture 80 may then be threaded (e.g., with a curved needle) into one of the open ends 136, through the suture lumen 134, out the other open end 136, and then tightened around the body structure BS. A suture knot 82 is then formed to hold the suture anchor 106 and associated portion of the lead body 104 against the body structure BS. The distance between the nerve cuff 102 and the suture anchor 106 may be such that the portion of the lead body 104 must be bent into a U-shape in order to position the nerve cuff 102 adjacent to the target nerve (e.g., the HGN). The nerve cuff 102 (FIGS. 2, 4 and 5) may be positioned around the target nerve before or after the leady body 104 is anchored to the body structure BS.

With respect to materials, the anchor body 128 may be formed from soft material (e.g., a durometer of 50-60 Shore A) with a strength similar to that of the body structure BS. If the anchor body material is too strong, it is possible that the suture 80 will tear through the body structure BS as the suture is tightened and, conversely, it is possible that the suture 80 will easily tear through the anchor body tissue contact portion 138 as the suture is tightened if the material is not strong enough. Silicone is one exemplary material that with suitable softness and strength properties. Silicone is also advantageous because radiopaque material such as barium sulfate can be mixed into silicone. Soft polyurethane is another suitable material.

It should also be noted here that the configuration of the exemplary anchor 106 reduces the likelihood of suture-based damage to the lead body 104. In particular, the location of the suture lumens 134, including the locations of the open ends 136, allows a suture (e.g., suture 80) to secure anchor 106 to the body structure without extending around lead body 104. Put another way, the lead body 104 will be located outside of the loop formed by the suture and the portion of the suture that is associated with the anchor 106, i.e. the portion within the lumen 134, is located between the lead body 104 and the body structure BS. As a result, in those instances where the suture 80 is tightened to the point at which it slices through the anchor body 128 (FIG. 10A), leaving a damaged tissue contact portion 138′ with a slice 139 between portions thereof, the suture 80 will not damage the lead body 104. The anchor 106 will simply be disconnected from the body structure BS.

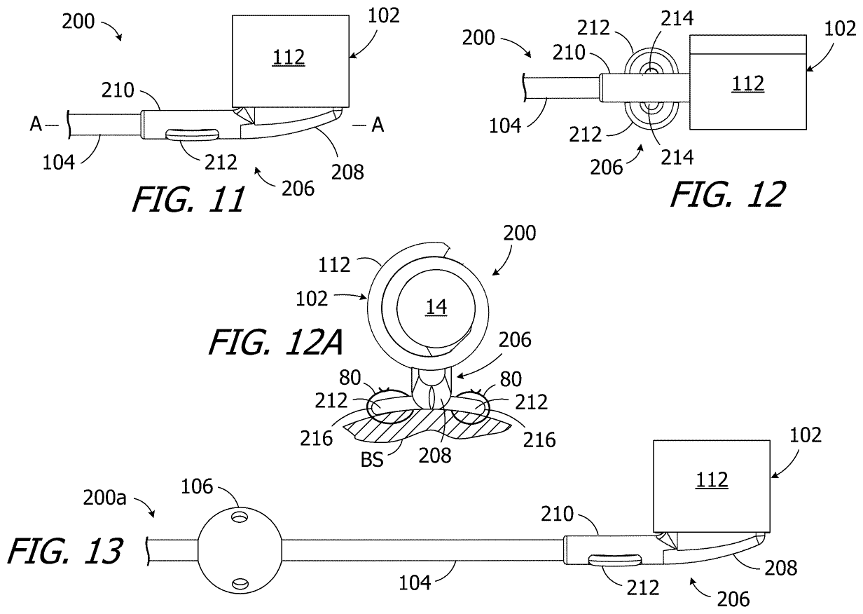

Turning to FIGS. 11 and 12, the exemplary electrode lead 200 illustrated therein is substantially similar to the electrode lead 100 and similar elements are represented by similar reference numerals. For example, the electrode lead 200 includes a nerve cuff 102 and a lead body 104. Here, however, electrode lead 200 includes a strain relief 206 where the lead body 104 (and wires 118 therein) meets the nerve cuff 102 that, in addition to conventional strain relief functionality, includes suture anchor functionality. As used herein, a “strain relief” is a mechanical device that improves structural integrity and protects cables, wires, and electrical connections from excessive tension, pulling, bending, or twisting forces. The exemplary strain relief 206 includes a base member 208 that is mounted onto the cuff body 112 and a tubular member 210 that extends from the base member 208 and that covers the end portion of the lead body 104. The exemplary strain relief 206 also includes one or more arcuate suture holders 212 (two in the illustrated embodiment), with suture openings 214, that extend outwardly from the tubular member 210. The tubular member 210 defines a central axis A and the arcuate suture holders 212 may be located symmetrically (as shown), i.e., are longitudinally aligned and are offset from one another by 180 degrees about the central axis A, or may be asymmetrically located. The arcuate suture holders 212 may be identical in shape and size (as shown) or may be different in size and/or shape. Suitable materials for the strain relief 206 include, but are not limited to, silicone and polyurethane. The base member 208, tubular member 210 and arcuate suture holders 212 may be formed from material having the same hardness or, in some instances, the arcuate suture holders 212 may be formed from harder material than the base member 208 and/or the tubular member 210. The strain relief 206 may also include internal braiding or wire that improves the strength of the arcuate suture holders 212.

One exemplary method of employing the arcuate suture holders 212 of the strain relief 206 is illustrated in FIG. 12A. Here, the sutures 80 pass through the suture openings 214 (FIG. 12), through the body structure BS (e.g., a tendon or muscle), and around the outer edges 216 of the arcuate suture holders 212. The location of the nerve cuff 102 that is positioned around, and on, a nerve (e.g. the HGN trunk 14) is thereby fixed at a location adjacent to the target location on the nerve.

In a manner similar to that described above, the configuration of the exemplary strain relief 206 reduces the likelihood of suture-based damage to the tubular member 210 and the lead body 104. In particular, and as shown in FIG. 12A, the location of the arcuate suture holders 212 allows a suture to secure the strain relief 206 to a body structure (e.g., a tendon or muscle) without extending around the tubular member 210 and the lead body 104. Put another way, the tubular member 210 and the lead body 104 will be located outside of the loops formed by the sutures 80 and the portion of the sutures that are associated with the anchor 206, i.e. the portions that extend through the arcuate suture holder openings 214, are located between the tubular member 210 (and associated portion of the lead body 104) and the body structure. As a result, in those instances where a suture is tightened to the point at which it slices through the arcuate suture holder 212, the suture will not damage the tubular member 210 or the lead body 104.

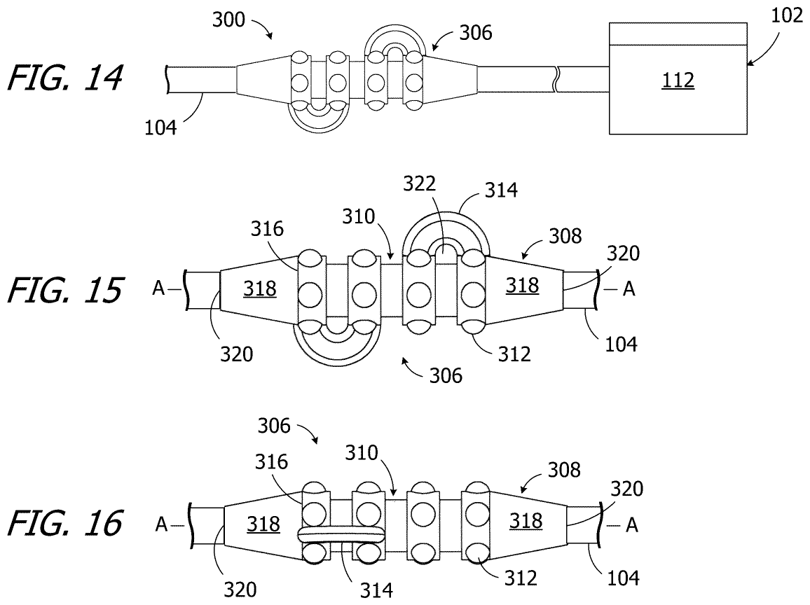

It should also be noted here that the strain relief 206 may be used in conjunction with the suture anchor 106 or the suture anchor 306 described below with references to FIGS. 14-16. Referring to FIG. 13, the electrode lead 200a is identical to the electrode lead 200 but for the inclusion of the suture anchor 106 on the lead body 104. Such as arrangement allows the surgeon to determine during the surgical procedure the most appropriate location (or locations) for anchoring.

Another exemplary electrode lead is generally represented by reference numeral 300 in FIG. 14. The exemplary electrode lead 300 illustrated therein is substantially similar to the electrode lead 100 and similar elements are represented by similar reference numerals. For example, the electrode lead 300 includes a nerve cuff 102 and a lead body 104. Here, however, the electrode lead 300 includes a suture anchor 306 in place of the suture anchor 106. The suture anchor 306 is configured to provide a plurality of the suture anchoring options.

Turning to FIGS. 15 and 16, the exemplary suture anchor 306 includes an anchor body 308, which defines a central axis A, and a plurality of different anchor features associated with the anchor body (i.e., grooves 310, protuberances 312 and arcuate suture holders 314) for use with one or more sutures. The anchor body 308 includes a cylindrical portion 316, two frusto-conical portions 318, and a lumen (not shown) for the lead body 104. The lumen extends through the anchor body 308 and defines open ends 320. The frusto-conical portions 318 do not include anchor features in the illustrated embodiment. Suitable materials for the anchor 306 include, but are not limited to, silicone and polyurethane

The exemplary grooves 310 extend inwardly from the outer surface of the anchor body cylindrical portion 316. The grooves 310 may extend completely around the central axis A (as shown) or partially around the central axis A. Although there are three grooves 310 in the illustrated embodiment, the number may be increased or decreased. The frusto-conical portions 318 may also include one or more grooves 310 in some implementations.

The exemplary protuberances 312 are hemispherical in shape and extend outwardly from the outer surface of the anchor body cylindrical portion 316. Other shapes may also be employed. There are also four sets of five protuberances 312, and the protuberances are equally spaced around the central axis A, in the illustrated embodiment. The number of protuberances 312 within each set, as well as the number of sets, may be increased or decreased. The frusto-conical portions 318 may also include protuberances 312 in some implementations.

The exemplary arcuate suture holders 314 define suture openings 322 and extend outwardly from the outer surface of the anchor body cylindrical portion 316. Although there are two arcuate suture holders 314 in the illustrated embodiment, the number of arcuate suture holders may be decreased or increased (e.g., to four). The arcuate suture holders 314 may also be located symmetrically (as shown) or asymmetrically about the central axis A, and may be longitudinally offset (as shown) or aligned. The arcuate suture holders 314 may also be identical in shape and size (as shown) or may be different in size and/or shape.

The presence of a variety of different anchoring features provides the surgeon with multiple options when it comes to the manner in which the suture (or sutures) will be deployed. For example, in those instances where one or more sutures are deployed around the anchor 306, one or more of the grooves 310 and/or the protuberances 312 may be used to guide and fix the longitudinal position(s) of the suture(s) on the anchor. In some instances, the sutures(s) may also pass through the openings 322 of the arcuate suture holders 314. Alternatively, sutures may pass through the openings 322 of the arcuate suture holders 314 without extending around the anchor body 308 in the manner described above.

With respect to the use of multiple loops, multiple sutures may be employed or a single suture may be looped multiple times. In either case, the use of multiple loops distributes the suture-induced stress on the anchor and reducing likelihood that the suture(s) will slice through the anchor or damage the associated body structure.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions. The inventions include any and all combinations of the elements from the various embodiments disclosed in the specification. The scope of the present inventions is limited solely by the claims set forth below.

1. An electrode lead, comprising:
an elongate lead body having a proximal region, a distal region and a plurality of electrical conductors;
a plurality of electrically conductive contacts associated with the distal region of the lead body and electrically connected to the electrical conductors; and
a suture anchor, on the distal region of the lead body, including an anchor body that defines a curved three-dimensional geometric exterior surface shape and a central axis that is coincident with a plane that passes through the anchor body, a lumen that extends through the anchor body in which a portion of the lead body is located, and at least one suture lumen that is entirely on one side of the plane and that has open ends on the exterior surface.

2. The electrode lead claimed in claim 1, wherein
the anchor body defines a spherical exterior surface shape.

3. The electrode lead claimed in claim 1, wherein
the anchor body is formed from soft material.

4. The electrode lead claimed in claim 1, wherein
the at least one suture lumen defines a curved shape in a plane perpendicular to the central axis.

5. The electrode lead claimed in claim 1, wherein
a tissue contact surface is located between the open ends of the at least one suture lumen.

6. The electrode lead claimed in claim 1, wherein
the anchor body is symmetrical about the central axis.

7. The electrode lead claimed in claim 1, wherein
the lumen in which the lead body is located is coaxial with the central axis.

8. The electrode lead claimed in claim 1, wherein
the at least one suture lumen comprises first and second suture lumens.

9. The electrode lead claimed in claim 8, wherein
the first and second suture lumens are located on opposite sides of the plane.

10. The electrode lead claimed in claim 9, wherein
the first and second suture lumens define respective midpoints; and
the midpoints of the first and second suture lumens are offset from one another by 180 degrees around the central axis.

11. The electrode lead claimed in claim 1, wherein
the first and second suture lumens define the same size and shape.

12. The electrode lead claimed in claim 1, further comprising:
a biologically compatible, elastic, electrically insulative nerve cuff body that is associated with the distal end of the lead body, is configured to be circumferentially disposed around a nerve, has a pre-set furled state that defines an inner lumen and is movable to an unfurled state;
wherein the plurality of electrically conductive contacts are carried by the nerve cuff body.

13. An electrode lead, comprising:
an elongate lead body having a proximal region, a distal region and a plurality of electrical conductors;
a nerve cuff, including a nerve cuff body and a plurality of electrically conductive contacts carried by the nerve cuff body and electrically connected to the electrical conductors, associated with the distal region of the lead body; and
a strain relief, associated with the lead body and the nerve cuff body, that includes a suture anchor.

14. The electrode lead claimed in claim 13, wherein
the strain relief includes a base member mounted to the nerve cuff body and a tubular member over a portion of the distal region of the lead body.

15. The electrode lead claimed in claim 14, wherein
the base member and the suture anchor are formed from materials having different hardnesses.

16. The electrode lead claimed in claim 14, wherein
the suture anchor is on the tubular member.

17. The electrode lead claimed in claim 1, wherein
the suture anchor comprises at least one arcuate suture holder with a suture opening.

18. The electrode lead claimed in claim 17, wherein
the suture anchor comprises first and second arcuate suture holders with respective suture openings.

19. The electrode lead claimed in claim 18, wherein
the tubular member defines a central axis; and
the first and second arcuate suture holders are longitudinally aligned and are offset from one another by 180 degrees about the central axis.

20. The electrode lead claimed in claim 1, further comprising:
a second suture anchor, located on the distal region of the lead body and proximal of the strain relief, including an anchor body that defines a curved three-dimensional geometric exterior surface shape and a central axis that is coincident with a plane that passes through the anchor body, a lumen that extends through the anchor body in which a portion of the lead body is located, and at least one suture lumen that is entirely on one side of the plane and that has open ends on the exterior surface.

21. The electrode lead claimed in claim 1, wherein
the nerve cuff body comprises a biologically compatible, elastic, electrically insulative nerve cuff body that is configured to be circumferentially disposed around a nerve, has a pre-set furled state that defines an inner lumen and is movable to an unfurled state.

22-30. (canceled)
